(19) United States
(12) Patent Application Publication
(10) Pub. No.: US 2021/0401023 A1
(43) Pub. Date: Dec. 30, 2021

(54) STEVIA PROTEIN

(71) Applicant: PURECIRCLE USA INC., Westchester, IL (US)

(72) Inventor: Avetik MARKOSYAN, Kuala Lumpur (MY)

(21) Appl. No.: 17/293,697

(22) PCT Filed: Oct. 31, 2019
(86) PCT No.: PCT/US2019/059240

§ 371 Date: May 13, 2021

Related U.S. Application Data
(60) Provisional application No. 62/768,392, filed on Nov. 16, 2018.

(51) Int. Cl. A23L 33/185; A23J 1/00; C07K 14/415; C12P 21/00

(52) U.S. Cl. CPC A23L 33/185; A23J 1/006; A23V 2002/00; C12P 21/00; C07K 14/415

(57) ABSTRACT

The invention relates to a Stevia protein obtained from Stevia plant material, and to a process for obtaining protein from Stevia plant material. The invention is directed to obtaining protein from Stevia rebaudiana plants, commonly referred to as simply “Stevia,” specifically the vegetative parts of the plant including the leaves and stems.

### BACKGROUND OF THE INVENTION

High intensity sweeteners possess a sweetness level many times exceeding that of sucrose. They are essentially non-caloric and used widely in manufacturing of diet and reduced calorie food. Although natural caloric sweeteners such as sucrose, fructose, and glucose provide the most desirable taste to consumers, they possess high calorie values. High intensity sweeteners do not affect the blood glucose level and provide little or no nutritive value.

Stevia rebaudiana Bertoni is a perennial shrub of the Asteraceae (Compositae) family native to certain regions of South America. The leaves of the plant contain from 10 to 20% of diterpene glycosides, which are around 150 to 450 times sweeter than sugar. The leaves have been traditionally used for hundreds of years in Paraguay and Brazil to sweeten local teas and medicines.

At present, there are more than 230 Stevia species with significant sweetening properties. The plant has been successfully grown under a wide range of conditions from its native subtropics to the cold northern latitudes.

The extract of Stevia rebaudiana plant contains a mixture of different sweet diterpene glycosides, which have a single base steviol and differ by the presence of carbohydrate residues at positions C13 and C19. These glycosides accumulate in Stevia leaves and compose approximately 10%-20% of the total dry weight. Typically, on a dry weight basis, the four major glycosides found in the leaves of Stevia are Dulcoside A (0.3%), Rebaudioside C (0.6-1.0%), Rebaudioside A (3.8%) and Stevioside (9.1%). Other glycosides identified in Stevia extract include Rebaudioside B, C, D, E, and F, Steviolbioside and Rubusoside.

Steviol glycosides have zero calories and can be used wherever sugar is used. They are ideal for diabetic and low-calorie diets.

On the other hand, it has to be noted that in process of manufacture along with steviol glycosides large amounts of other constituents of Stevia plant are also extracted with water. These other constituents are mainly separated during downstream processing and discarded into environment.

Little is known about these other constituents of Stevia rebaudiana plant. Few authors reported phenolics compounds, free amino acids etc. however the information about the identity of those constituents remains scarce and their possible uses in foods, beverages and other consumables is not described (Karaköse, et al, 2011, 2015; Wölwer-Rieck, 2012; Periche et al, 2014).

If accomplished at a large scale, processing the other extracted constituents of the Stevia plant can provide significant economic, and environmental benefits as it can provide an opportunity for inclusion of the whole Stevia plant into the food chain, creating practically waste-free Stevia processing.

Therefore, there is a need to develop a method to effectively isolate those constituents, including proteins, from the Stevia rebaudiana plant and use them as ingredients in different consumables.

### SUMMARY OF THE INVENTION

The invention is directed to obtaining protein from Stevia rebaudiana plants, commonly referred to as simply “Stevia,” specifically the vegetative parts of the plant including the leaves and stems. In particular, Stevia leaves and stems, and their related spent biomass remaining after extraction of steviol glycosides from the leaves, are used to obtain protein.

In one embodiment, the invention is directed to a Stevia protein obtained from Stevia plant material. The Stevia plant material may be vegetative Stevia material.

In one embodiment, the invention is directed to a method for making a Stevia protein, including providing Stevia plant material and extracting protein from the Stevia plant material.

In one embodiment, the invention is directed to a method for making a Stevia protein, including providing Stevia plant material, subjecting the Stevia plant material to microbial treatment to produce protein, and separating the protein from the Stevia plant material.

### DETAILED DESCRIPTION

The protein from Stevia rebaudiana plants can be obtained in many ways from the vegetative Stevia material, such as by extraction, or as a substrate for microbial fermentation. Obtaining protein from the vegetative Stevia material provides a valuable use of the material. In certain embodiments, steviol glycosides are first extracted from the Stevia plant leaves, and then the remaining vegetative Stevia material is processed to obtain Stevia. In other embodiments, vegetative Stevia material is used directly to obtain protein, without extracting steviol glycosides from the leaves. Any method that can be used to obtain protein from vegetative plant material is contemplated by this invention. For example, any method to produce leaf protein concentrate is contemplated.

The vegetative Stevia material can also be used as a feedstock for microorganisms capable of producing proteins. Examples of such microorganisms include but are not limited to Escherichia coli, Corynebacterium, Pseudomonas fluorescens, Saccharomyces cerevisiae, Pichia pastoris, Aspergillus species, Trichoderma species, Myceliopthora thermophila, Baculovirus-infected cells, Leishmania species, Rhizopus sp., Aspergillus sp., Saccharomyces sp., Lactobacillus sp., Rhizopus oryzae, Aspergillus niger, Saccharomyces bulardii, Lactobacillus plantarum, Lactobacillus rhamnosus, and the like. This invention further contemplates the use of cell-free systems for producing proteins, using RNA polymerase, ribosomes, tRNA and ribonucleotides obtained from microbial cells. Any cellular or cell-free process to produce proteins is contemplated by this invention.

The preparation of feedstock using Stevia plant materials is described in U.S. Pat. Nos. 8,530,527 and 9,771,434, both of which are incorporated herein by reference in their entireties.

The Stevia protein thus obtained may optionally be further processed for purification, drying, decolorizing, and the like, and may be delivered in various forms, such as a powder, liquid, gel, granules, tablet, capsule, caplet, wafer, agglomerates, and the like.

The Stevia protein thus obtained can be used in many ways, including as a food ingredient, a flavor, a consumable, a nutraceutical, a dietary supplement, a coating, a cosmetic preparation, a pharmaceutical preparation, a healthcare product, a processing aid such as a catalyst, and the like. The Stevia protein may be combined with any other material in any form to make a composition. Any conventional use of proteins is contemplated by this invention.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

What is claimed is:

1. A Stevia protein obtained from Stevia plant material.

2. The Stevia protein of claim 1, wherein the Stevia plant material is vegetative Stevia material.

3. A method for making a Stevia protein, comprising the steps of:
a. Providing Stevia plant material; and
b. Extracting protein from the Stevia plant material.

4. A method for making a Stevia protein, comprising the steps of:
a. Providing Stevia plant material;
b. Subjecting the Stevia plant material to microbial treatment to produce protein; and
c. Separating the protein from the Stevia plant material.
